SEQUENCE LISTING (6 sequences)

SEQ ID NO: 1 - DNA, 30 bp, Artificial Sequence
  Synthetic
gcactgagttcatttgctataagtagagtt

SEQ ID NO: 2 - DNA, 19 bp, Artificial Sequence
  Synthetic
gctgctcctgctgaacttc

SEQ ID NO: 3 - DNA, 25 bp, Artificial Sequence
  Synthetic
ccgctaccaagtcaagaagctttca

SEQ ID NO: 4 - DNA, 25 bp, Artificial Sequence
  Synthetic
actcctgcagtaatccactttccaa

SEQ ID NO: 5 - DNA, 24 bp, Artificial Sequence
  Synthetic
ggaatggcgtgtgtgttagccaaa

SEQ ID NO: 6 - DNA, 24 bp, Artificial Sequence
  Synthetic
tcacacaggaaacagctatgacca

(19) United States
(12) Patent Application Publication
(10) Pub. No.: US 2011/0256604 A1
(43) Pub. Date: Oct. 20, 2011

(54) GENERATION OF ASPOROGENOUS SOLVENTOGENIC CLOSTRIDIA

(75) Inventors: Eleftherios T. Papoutsakis, Newark, DE (US); Daniel R. Hess, New Castle, DE (US); Bryan P. Tracy, Wilmington, DE (US)

(73) Assignee: University of Delaware, Newark, DE (US)

(21) Appl. No.: 13/009,215

(22) Filed: Jan. 19, 2011

Related U.S. Application Data
(60) Provisional application No. 61/296,229, filed on Jan. 19, 2010.

(51) Int. Cl. C12P 1/04; C12N 15/74; C12N 1/21

(52) U.S. Cl. 435/170; 435/252.3; 435/471

(57) ABSTRACT

Expression of the SpoIIE gene in a solventogenic Clostridium cell is silenced and sporulation is abolished. The cell exhibits increased production of a solvent, such as butanol, relative to a wild-type solventogenic Clostridium cell and can be used for industrial-scale production of a chemical product. A method includes silencing the SpoIIE gene of the Clostridium cell via a homologous recombination method in which a resolvase gene is expressed. Another method includes increasing solvent production in bacterial cells by inoculating the cells with an inoculum of exponentially growing cells, wherein expression of a sporulation gene in the bacterial cells is inhibited or silenced and the cells of the inoculum are in a post-exponential phase of growth prior to inoculation.

### CROSS-REFERENCE TO RELATED APPLICATIONS

This application claims priority to provisional application Ser. No. 61/296,229, filed Jan. 19, 2010, which is incorporated herein by reference in its entirety and for all purposes.

### STATEMENT OF GOVERNMENT SUPPORT

Research leading to the disclosed inventions was funded, in part, by the National Science Foundation Grant No. 0853490. Accordingly, the United States Government may have certain rights in this invention.

### FIELD OF THE INVENTION

This invention relates generally to the inactivation of sporulation in solventogenic Clostridium strains. In particular, embodiments of the invention relate to enhanced butanol production in Clostridium cells that have silenced expression of the SpoIIE gene.

### BACKGROUND OF THE INVENTION

Various Clostridium strains have had a long industrial history in which they were used in the anaerobic ABE (Acetone-Butanol-Ethanol) fermentation, which was a profitable industrial process up until the 1950's when the petrochemical process became dominant (Jones, D. T. and D. R. Woods, Acetone-butanol fermentation revisited. Microbiol. Rev, 1986. 50(4): p. 484-524; Moo-Young, M., Comprehensive biotechnology: the principles, applications, and regulations of biotechnology in industry, agriculture, and medicine, 1st ed. 1985, Oxford, New York: Pergamon Press). The main product of interest from ABE fermentation is butanol, although acetone and other side products are also of interest. Currently, butanol is widely used as an industrial solvent and in the future could potentially be used as a biofuel. Butanol has several superior chemical properties over ethanol, as it has higher energy content per unit mass, is less volatile and hydrophilic, and is more miscible with hydrocarbons.

Traditionally, the ABE fermentation was carried out in a batch mode, in which the cells initially produced butyric and acetic acid. Once a threshold concentration of undissociated butyric acid had built up, the cells switched over to solvent production (Jones, D. T. and D. R. Woods; Dürre, P., New insights and novel developments in clostridial acetone/butanol/isopropanol fermentation, Applied Microbiology and Biotechnology, 1998. 49(6): p. 639-648; Woods, D R., The genetic engineering of microbial solvent production. Trends Biotechnol, 1995. 13(7): p. 259-64.). Acetone, butanol, and ethanol can be produced directly from feedstock, and can also be produced by re-assimilating butyric and acetic acid to produce butanol and ethanol, respectively, with acetone produced as a by-product of the re-assimilation process. Currently, this process cannot compete with the petrochemical production of butanol because of low butanol titers, the relatively low selectivity for butanol (ratio of butanol to other products), and the low productivity of batch fermentations. Typically, the ABE fermentation rarely exceeds 12-13 g/L of butanol (Moo-Young, M.; Marlatt, J. A. and R. Datta, Acetone-Butanol Fermentation Process-Development and Economic-Evaluation, Biotechnology Progress, 1986. 2(1): p. 23-28), but various economic analyses estimate that by increasing a final butanol concentration to 19 g/L, the separation costs can be cut in half (Moo-Young, M.; Marlatt et al.; Dadgar, A. M. and G. L. Foutch, Improving the Acetone-Butanol Fermentation Process with Liquid-Liquid-Extraction. Biotechnology Progress, 1988. 4(1): p. 36-39; Lenz, T. G. and A. R. Moreira, Economic-Evaluation of the Acetone-Butanol Fermentation, Industrial & Engineering Chemistry Product Research and Development, 1980. 19(4): p. 478-483).

In wild-type (WT) cultures of solventogenic Clostridium strains, solvent formation is coupled to sporulation, in that when one is induced, so is the other. From a bioprocessing perspective, sporulation is an undesirable cellular program because it is energy intensive and can inhibit cellular growth and dilute the population of solvent forming cells. It is therefore desirable to decouple solvent formation from sporulation in order to enhance solvent production.

### SUMMARY OF THE INVENTION

An embodiment of the present invention provides a Clostridium cell in which expression of the SpoIIE gene of the Clostridium cell is silenced and sporulation by the cell is abolished. Preferably, the Clostridium cell exhibits increased production of a solvent, such as butanol, relative to a wild-type Clostridium cell of the same strain. The Clostridium cell can be used for industrial-scale production of a chemical product, such as butanol, butyric acid, acetoin, butanediol, or propanol.

In other embodiments, the present invention provides methods for abolishing sporulation in a solventogenic Clostridium cell comprising silencing the expression of the SpoIIE gene of the cell. Preferably, the method comprises transforming the cell with a vector comprising a resolvase gene and a nucleic acid that disrupts the function of the SpoIIE gene following homologous recombination, wherein the nucleic acid integrates into the genome of the Clostridium cell.

Additional embodiments of the present invention provide methods for increasing solvent production in bacterial cells in which expression of a sporulation gene in the bacterial cells has been inhibited or silenced, comprising inoculating a medium with an inoculum of the bacterial cells, wherein the bacterial cells of the inoculum are in a post-exponential phase of growth prior to inoculation.

### BRIEF DESCRIPTION OF THE FIGURES

FIG. 1: Possible scenarios for integration on a chromosome. First, the replicating knock-out plasmid would undergo a single crossover event and integrate within the 1st homologous region or the 2^{nd} region. This integration could be stable, or it can undergo further crossovers. The integration could undergo a plasmid excision crossover, in which case, the wildtype gene is restored. The integration could also undergo a double crossover, in which the plasmid backbone (bb) would be excised leaving only the thiamphenicol marker disrupting the gene.

FIG. 2: Expected product sizes from various integration scenarios using Primer Set 1. Primer Set 1 includes SpoIIE-KO-F, a forward primer for the 5′ flanking region, and CM/TH-R, a reverse primer for the CM/TH marker.

FIG. 3: Expected product sizes from various integration scenarios using Primer Set 2. Primer Set 2 includes CM/TH-F, a forward primer for the CM/TH marker, and SpoIIEKO-R, a reverse primer for the 3′ flanking region.

FIG. 4: Phase contrast microscopy of WT and SpoIIEKO. SpoIIEKO cells display no significant differentiation phenotypes, even after 72 hours of growth.

FIG. 5: Butanol formation of SpoIIEKO and WT after inoculating at different times. Butanol formation of SpoIIEKO is dependent on inoculation time whereas butanol production in WT cultures are independent of time of inoculation.

### DETAILED DESCRIPTION OF THE INVENTION

All of the major transcription factors involved in sporulation in B. subtilis have been identified in Clostridium acetobutyllcum ATCC824 (GenBank#AE001437, Refseq: NC_{—}003030), including σ^{F} (“sigF”) (Milling, J., et al., Genome sequence and comparative analysis of the solvent-producing bacterium Clostridium acetobutylicum. J Bacteriol, 2001. 183(16): p. 4823-38; Paredes, C. J., K. V. Alsaker, and E. T, Papoutsakis, A comparative genomic view of clostridial sporulation and physiology. Nat Rev Microbiol, 2005, 3(12): p. 969-78; Santangelo, J. D., et al., Sporulation and time course expression of sigma-factor homologous genes in Clostridium acetobutylicum, Ferns Microbiology Letters, 1998. 161(1): p. 157-164; Sauer, U., et al., Sigma factor and sporulation genes in Clostridium. FEMS Microbiol Rev, 1995, 17(3): p. 331-40; Sauer, U., et al., Sporulation and primary sigma factor homologous genes in Clostridium acetobutylicum. J Bacteriol, 1994. 176(21): p. 6572-82; Wong, J., C. Sass, and G. N. Bennett, Sequence and arrangement of genes encoding sigma factors in Clostridium acetobutylicum ATCC 824, Gene, 1995. 153(1): p. 89-92).

Encoded by the sigF (CAC2306) gene, the sigF protein shares 42.7% identity with the sigF protein in B. subtilis, and there is evidence that, in accordance with the Bacillus model, it is the first sporulation-specific sigma factor to become active when sporulation is initiated (Alsaker, K. V. and E. T, Papoutsakis, Transcriptional program of early sporulation and stationary-phase events in Clostridium acetobutylicum. Journal of Bacteriology, 2005. 187(20): p. 7103-7118; Jones, S. W., et al., The transcriptional program underlying the physiology of clostridial sporulation, Genome Biol, 2008. 9(7): p. R114). SpoIIE is a sporulation-specific membrane-bound serine phosphatase in endospore-forming, Gram-positive organisms, and according to the Bacillus subtilis sporulation model, indirectly activates sigF (Hilbert, D. W. and P. J. Piggot, Compartmentalization of gene expression during Bacillus subtilis spore formation. Microbiol. Mol Biol Rev, 2004. 68(2): p. 234-62; Piggot, P. J. and D. W. Hilbert, Sporulation of Bacillus subtilis. Curr Opin Microbiol, 2004. 7(6): p. 579-86; Steil, L., et al., Genome-wide analysis of temporally regulated and compartment-specific gene expression in sporulating cells of Bacillus subtilis, Microbiology, 2005. 151(Pt 2): p. 399-420; Stragier, P. and R. Losick, Molecular genetics of sporulation in Bacillus subtilis. Annu Rev Genet, 1996. 30: p. 297-41).

When solvent production is initiated in C. acetobutyclium ATCC824 (e.g., when a threshold concentration of undissociated butyric acid has been reached), chief among the genes upregulated for solvent production are the genes in the sol operon (adhE1-ctfA-ctfB, CAP0162-4) and adc (CAP0165) (Dürre, P. et al., Transcriptional regulation of solventogenesis in Clostridium acetobutylicum. Journal of Molecular Microbiology and Biotechnology, 2002 4(3): p. 295-300; Girbal, L. and P. Soucaille, Regulation of solvent production in Clostridium acetobutylicum. Trends in Biotechnology, 1998. 16(1): p. 11-16; Thormann, K., et al., Control of butanol formation in Clostridium acetobutylicum by transcriptional activation. Journal of Bacteriology, 2002. 184(7): p. 1966-1973). All of these genes are located on the 192 kb pSOL1 megaplasmid and have been found to be regulated by Spo0A (Dürre, P. et al.; Girbal, L. and P. Soucaille; Thormann, K., et al.; Ravagnani, A., et al., SpoOA directly controls the switch from acid to solvent production in solvent-forming clostridia, Molecular Microbiology, 2000. 37(5): p. 1172-1185). This discovery provided the link between solventogenesis and sporulation, since in the Bacillus model of sporulation, Spo0A is the master regulator of the sporulation cascade. The central role of Spo0A in both solventogenesis and sporulation was demonstrated in C. acetobutylicum by the Sp0A mutant strain SK01, in which Spo0A expression was silenced. In SK01, solventogenesis was essentially silenced, acetone and butanol production were reduced to 2 and 8% of WT levels, respectively, and no identifiable spore differentiation phenotypes were observed (Harris, L. M., N. E. Welker, and E. T. Papoutsakis, Northern, morphological, and fermentation analysis of spo0A inactivation and overexpression in Clostridium acetobutylicum ATCC 824. Journal of Bacteriology, 2002. 184(13): p. 3586-3597).

In B. subtilis, after activation, Spo0A influences the expression of 121 genes, including sigF and sigE, and regulators of the next stages of sporulation in the prespore and mother cell, respectively (Molle, V., et al., The SpoOA regulon of Bacillus subtilis. Molecular Microbiology, 2003. 50(5): p. 1683-1701). Initially, the stage II sporulation protein E (i.e., SpoIIE) dephosphorylates the anti-anti-sigma factor, SpoIIAA. In its active form, SpoIIAA binds the anti-sigma factor SpoIIAB, thus releasing sigF so it can direct the processing of pro-sigE into active sigE. Both of these factors then go on to regulate the expression of sigG in the developing endospore, and sigE and sigG regulate the expression of sigK, the last of the sigma factors, in the mother cell. All of these sigma factors have been identified in C. acetobutylicum, and in a transcriptional profiling of a typical batch fermentation using genomic microarray, expression of all the sigma factors was observed except sigK. More importantly, based on deduced activity plots, it appears that the order of activation of the various factors follows the same pattern as in B. subtilis (i.e. spo0A to sigF to sigE to sigG). Currently, it is not known what influence, if any, these sporulation-specific sigma factors have on solventogenesis.

An embodiment of the present invention provides a solventogenic Clostridium cell in which expression of the SpoIIE gene is silenced and sporulation by the Clostridium cell is abolished. A Clostridium cell according to the present invention is solventogenic and is preferably selected from the group consisting of C. acetobutylicum, C. beijerinckii, C. saccharoperbutylacetonicum, and C. madisonii; and most preferably the Clostridium cell is C. acetobutylicum. As used herein, the “SpoIIE gene” refers to a nucleic acid (e.g., DNA) sequence that comprises coding sequence(s) necessary for the production of the SpoIIE protein; the SpoIIE protein can be encoded by the full length of the nucleic acid sequence or by any portion of the nucleic acid sequence. As used herein, when a sporulation gene (e.g., the SpoIIE gene) in a cell is described as being “silenced,” the gene has been rendered inactive, i.e., it is not expressed by the cell. As used herein, a sporulation gene that has been “inhibited” or “downregulated” is a gene that is only partially inactivated such that sporulation is not abolished but is merely decreased. As used herein, when sporulation by a cell is described as being “abolished,” “arrested,” or “non-sporulating,” the cell never sporulates throughout the life of the cell in culture. According to embodiments of the present invention the expression of the SpoIIE gene is not merely inhibited or downregulated, but is silenced, and sporulation is not merely inhibited or downregulated, but is abolished and the cell is non-sporulating. Thus, the activity of the SpoIIE protein in Clostridia strains is preferably reduced by 100%.

In one embodiment, as described in the example below, cells in which SpoIIE had been silenced were observed in phase-contrast microscopy. Even at 72 hours, no free spores or spore-forming phenotypes were observed. The only spore differentiation activity that was observed was the condensing of DNA at one or both ends of the cell; however, the next step in spore differentiation, septation, was never observed, and none of the cells exhibited a spore-forming phenotype or produced spores. In addition, after 115 hours, none of the cells in which SpoIIE had been silenced survived chloroform treatment, whereas the wild-type Clostridium cells of the same strain survived the chloroform treatment and produced spores, indicating that even at 115 hours in culture, cells according to an embodiment of the invention are non-sporulating and, preferably, an asymmetric septation event of spore differentiation does not occur in the cells throughout their life in culture (i.e., the spore differentiation process is arrested prior to asymmetric septation).

Applicants are the first to silence SpoIIE expression in a solventogenic Clostridium species. By silencing the expression of SpoIIE, applicants created a non-sporulating strain of Clostridium with enhanced butanol production capabilities. Compared to a wild-type Clostridium cell of the same strain, the SpoIIE knockout strain (i.e., the strain in which expression of SpoIIE was silenced) produced more butanol and exhibited no differentiation phenotype, i.e., no sporulation. Previous attempts have been made to decouple solventogenesis from sporulation; however, previous strategies were only able to downregulate (but not abolish) the expression of SpoIIE (Bennett, G. N. and M. C. Scotcher, U.S. patent application Ser. Nos. 11/173,542 and 12/196,164; Scotcher, M. C. and G. N. Bennett, SpoIIE regulates sporulation but does not directly affect solventogenesis in Clostridium acetobutylicum ATCC 824. Journal of Bacteriology, 2005. 187(6): p. 1930-1936). By downregulating SpoIIE using antisense RNA, previous investigators were able to decrease sporulation but never abolished sporulation. From a bioprocessing perspective, sporulation is an undesirable cellular program because it is energy intensive and can inhibit cellular growth and dilute the population of solvent forming cells. By abolishing sporulation, as opposed to merely inhibiting sporulation, applicants created a strain of Clostridium which can provide enhanced solventogenic capabilities in bioprocessing.

According to another aspect of the invention, a Clostridium cell in which the SpoIIE gene is silenced produces more solvent (e.g., butanol) than a wild-type Clostridium cell of the same strain. The solvent is selected from the group consisting of, for example, butanol, butyric acid, acetoin, butanediol, and propanol. The solvent produced by the Clostridium cell is preferably butanol. In one embodiment, a Clostridium cell produces between about 1% and about 100% more solvent than a wild-type Clostridium cell of the same strain, preferably between about 15% and about 20% more solvent than a wild-type Clostridium cell of the same strain. In the example below, a Clostridium cell according to the present invention produces about 17% more solvent than the wild-type strain. In one embodiment, an increase in solvent production compared to a wild-type strain occurs after about 120 hours of growth of the Clostridium cell.

According to embodiments of the present invention, a solventogenic Clostridium cell in which expression of the SpoIIE gene is silenced, and in which sporulation is abolished, can be used for industrial-scale production of butanol or other chemical products produced by Clostridium strains, such as butyric acid, acetoin, butanediol, and/or propanol. For example, Clostridia cells according to the present invention can be used for the industrial production of biofuels, in the fermentative production of chemical feedstocks, or in the fermentative production of bulk chemicals, such as butanol.

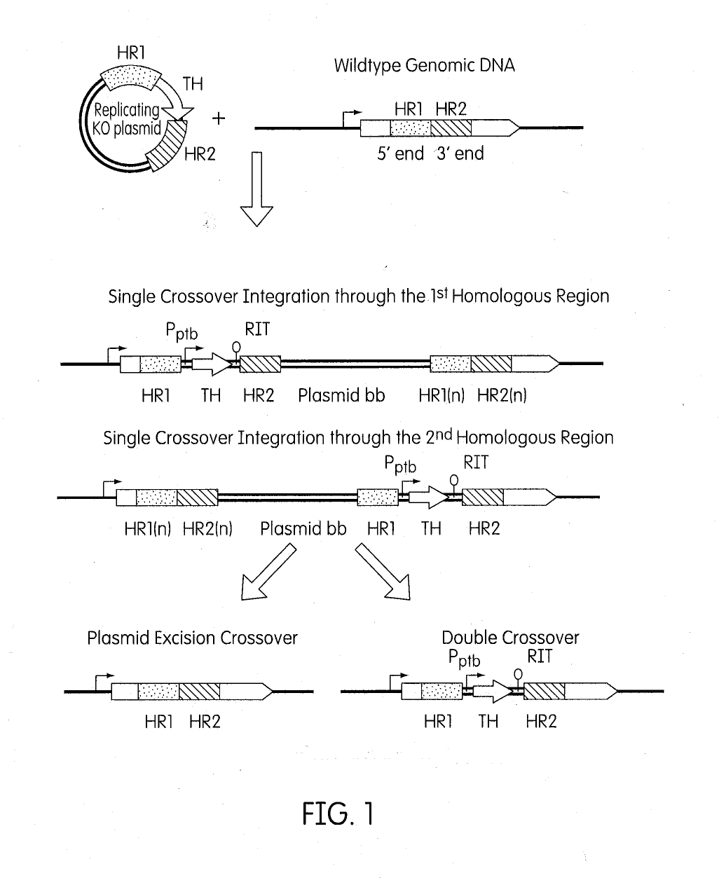

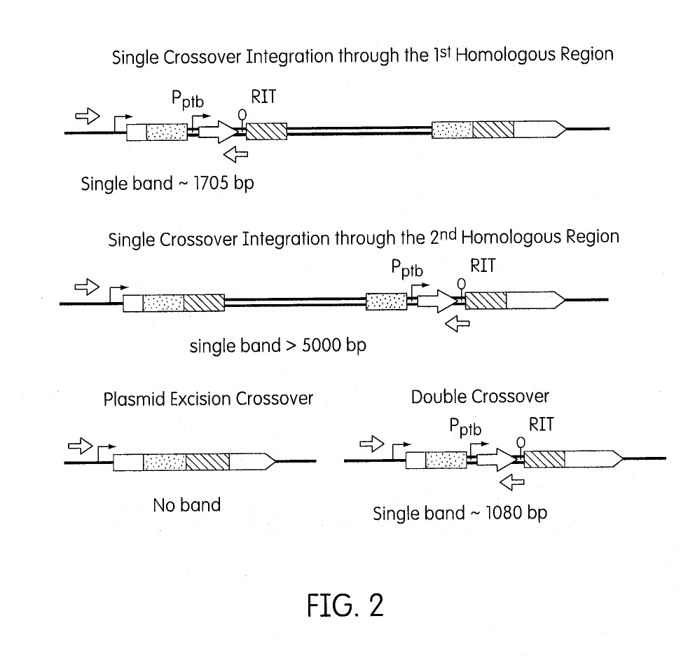

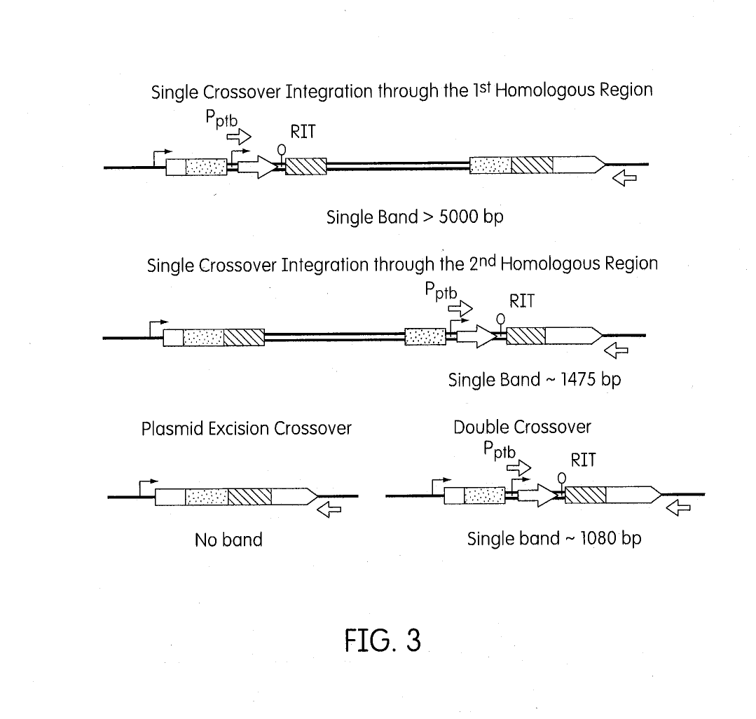

Embodiments of the present invention provide methods for silencing the SpoIIE gene in a solventogenic Clostridium cell, wherein sporulation by the cell is abolished, preferably by homologous recombination, thereby producing a cell in which sporulation is abolished. Preferred methods are described in U.S. Patent Publication No. 2010/0047890 and U.S. Patent Publication No. 2010/0075424, which are incorporated herein in their entirety. In one embodiment, the SpoIIE gene is silenced by transforming the cell with a plasmid comprising a nucleic acid that silences the function of the SpoIIE gene (e.g., by mutating the SpoIIE gene) following homologous recombination whereby the nucleic acid integrates into the genome of the Clostridium cell. Preferably, the nucleic acid that silences the function of the cell's SpoIIE gene (i.e., knocks out the SpoIIE gene) is a SpoIIE gene that has been altered (e.g., mutated), such that the altered SpoIIE gene is not able to express the SpoIIE protein. In some embodiments, methods according to the present invention comprise expressing a resolvase protein in the Clostridium cell. Preferably, the resolvase gene is the Bacillus subtilis recU gene BSU22310, although other resolvase genes may be used. Methods according to the present invention include constructing a vector (e.g., a plasmid) comprising a resolvase gene and an altered (e.g., mutated) SpoIIE gene and transforming a Clostridium cell with the vector. The resolvase gene is expressed in the Clostridium cell and the altered SpoIIE gene is integrated into the genome (i.e., the chromosomal DNA) of the Clostridium cell via homologous recombination by, for example, a double crossover event or a single integration event. Possible scenarios for integration on a chromosome are shown, for example, in FIGS. 1-3. In a preferred embodiment, the resulting Clostridium cell is a gene disruption mutant comprising a silenced SpoIIE gene (i.e., the SpoIIE gene is inactive) and sporulation in the cell is abolished.

Applicants discovered that, surprisingly, enhanced solvent production (e.g., increased butanol production) by bacterial cells (e.g., Bacillus cells or Clostridium cells) in which a sporulation gene has been inhibited, downregulated, or silenced, compared to wild-type bacterial cells of the same strain, can be dependent upon the age of the inoculum of the growing bacterial cells. In particular, it was discovered that if an older culture is used as the inoculum (i.e., an inoculum that has passed the exponential phase of growth and has entered an early to late stationary phase), cells in which expression of SpoIIE is inhibited or silenced can produce increased levels of solvent (e.g., butanol). The typical protocol for starting a new or larger culture of bacterial cells is to inoculate a medium with a culture that is in its exponential phase of growth. This normally reduces the lag time of the newly inoculated culture. Applicants observed that the typical inoculation protocol (i.e., inoculation with a culture in its exponential phase of growth) resulted in marginal solvent production in the inoculated culture. Surprisingly, however, when early to late stationary phase (i.e., post-exponential phase) inoculums were used, solvent production was enhanced, and was superior to the wild-type cultures. Without being bound to any theory, it is believed that SpoIIE inactivation prevents cells that have entered endospore formation from re-populating a fresh culture since they are terminally stalled at an early stage of endospore formation. This gives a growth advantage to cells that did not commit to endospore sporulation, enabling them to re-populate the inoculated culture. This is coupled with the possibility of an epigenetic inheritance event that enhances solvent production, and is associated with cells that did not commit to endospore formation. Moreover, C. acetobutylicum cultures and mutant cultures generally do not sporulate at 100% frequency (Jones, S. W., et al., The transcriptional program underlying the physiology of clostridial sporulation, Genome Biol, 2008. 9(7): p. R114), which supports the hypothesis that only a percentage of a culture enters endospore formation.

An embodiment of the present invention provides a method for increasing solvent production in bacterial cells (i.e., a bacterial cell culture), comprising inoculating a medium with an inoculum of bacterial cells (e.g., Bacillus cells or Clostridium cells), wherein the expression of a sporulation gene in the bacterial cells is inhibited or silenced, and wherein the bacterial cells of the inoculum are in a post-exponential phase of growth (e.g., an early to late stationary phase). The medium may be fresh (i.e., containing no bacterial cells), so that a new culture is started by inoculating the fresh medium. Alternatively, the medium may already contain a culture of bacterial cells, so that the culture is enlarged by inoculating with additional bacterial cells. Preferably, the cells of the inoculum have not entered endospore formation and have otherwise not committed to endospore formation. In a preferred embodiment, the inoculum of exponentially growing cells has been growing for at least about 20 hours prior to inoculating the bacterial cells, preferably between about 30 hours to about 75 hours, more preferably between about 50 hours and about 60 hours, and most preferably about 56 hours. In an exemplary embodiment, the bacterial cells are C. acetobutylicum cells, the SpoIIE gene in the cells is silenced, and sporulation by the Clostridium cells has been abolished.

Additional embodiments of the present invention provide methods for producing a chemical product. The chemical product is preferably selected from the group consisting of butanol, butyric acid, acetoin, butanediol, and propanol. A method according to the present invention comprises contacting a Clostridium cell or culture of Clostridium cells according to the present invention (i.e., solventogenic Clostridium cells in which the SpoIIE gene has been silenced) with a feedstock. Preferably, the feedstock comprises a substrate for the cells to produce the chemical product(s), such as a carbohydrate. The feedstock may also comprise substances such as butyric and/or acetic acid.

The following example is provided to describe the invention in greater detail. It is intended to illustrate, not limit, the invention.

### Example 1

Construction of spoIIE targeted gene disruption plasmid. A targeted gene disruption plasmid was generated using the same approach as described in U.S. Patent Publication No. 2010/0047890 and U.S. Patent Publication No. 2010/0075424, which are incorporated herein in their entirety. For the C. acetobutylicum spoIIE gene (CAC3205) targeted plasmid, the disrupted spollE gene fragment was constructed in the pCR®8/GW/TOPO®TA cloning plasmid from Invitrogen (Carlsbad, Calif.). A 587 bp region of the spoIIE gene was PCR amplified with AmpliTaq Gold® DNA polymerase from Applied Biosystems (Foster City, Calif.) and the SpoIIE-F (SEQ ID NO 1)/SpoIIE-R (SEQ ID NO 2) primer set (see Table 2), and then cloned into the pCR®8/GW/TOPO®TA cloning plasmid and One Shot® TOP10 E. coli via manufacturer suggestions. The resulting plasmid is called pCR8-SpoIIE. The spoIIE gene fragment was then disrupted in approximately the middle of the gene fragment via a Dra1 endonuclease (New England Biolabs, Ipswich, Mass.) digestion. The linear plasmid was then dephosphorylated using Antarctic Phosphatase (NEB). An antibiotic cassette was cloned into the linear plasmid via NEB Quick Ligation™ Kit and cloned into Invitrogen One Shot® TOP10 E. coli. The antibiotic cassette for the spoIIE disruption was a modified chloramphenicol/thiarnphenicol (CM/TH) marker described in Sillers, R., et al., Metabolic engineering of the non-sporulating, non-solventogenic Clostridium acetobutylicum strain M5 to produce butanol without acetone demonstrate the robustness of the acid-formation pathways and the importance of the electron balance, Metab Eng, 2008. 10(6); p. 321-32. The resulting plasmid is designated pCR8-SpoIIE/CM. The SpoIIE/CM gene disruption cassette was then recombined into a destination vector using Invitrogen's Gateway® system. The destination vector contains a Gram-positive origin of replication, a Gram-negative origin of replication, an MLSr cassette, and the resolvase recU gene (BSU22310) under a strong promoter (as described in U.S. Patent Publication No. 2010/0075424). The final replicating, spoIIE targeted plasmid is called pKORSpoIIE. The plasmid was next site specifically methylated to avoid degradation by the clostridial endonuclease CAC8241 by shuttling the plasmid through E. coli ER2275 pAN2. pAN2 contains a gene encoding for the site-specific methyltransferase. Finally, the methylated pKORSpoIIE was transformed into C. acetobutylicum via a previously reported electroporation protocol (Mermeistein, L. D., et al., Expression of cloned homologous fermentative genes in Clostridium acetobutylicum ATCC 824. Biotechnology (N Y), 1992. 10(2): p. 190-5). The strains and plasmids employed are shown in Table 1.

TABLE 1

| Strain or Plasmid | Relevant Characteristics | Source |
|---|---|---|
| Strain | | |
| E. coli One Shot ® | Invitrogen competent cells | Invitrogen |
| Chemically | | |
| E. coil ER2275 | recA lacZ mcrBC | N E B |
| C . acetobutylicum | type strain | ATCC |
| FKO | ATCC824 sigF::Th^{r} | this study |
| Plasmids | | |
| pAN2 | Amp^{r}; carries the φ3T1 gene | Tomas, C. |
| pCR8 ®/GW/TOPO ®TA | Sp^{r}; topoisomerized; ori | Invitrogen |
| pCR8-SpoIIE | pCR8 ®/GW/TOPO ®TA with | this study |
| | spoIIE fragment | |
| pCR8-SpoIIE/CM | pCR8 ®/GW/TOPO ®TA with | this study |
| | SpoIIE::Th^{r} | |
| pKORSpoIIE | MLS^{r}; repL ori; recU under thl | this study |
| | promoter; spoIIE::Th^{r} | |

Generation of spoIIE disruption mutants. Disruption mutants were generated using the same approach as described in U.S. Patent Publication No. 2010/0047890. Briefly, transformants were vegetatively transferred every 24 hrs for 10 days via replica plating on solid 2×YTG plates supplemented with thiamphenicol (TH) at 5 μg/ml. After ten days, the cells were again vegetatively transferred for an additional six days under no antibiotic selection to facilitate plasmid curing (i.e., to lose the plasmid). After six days of curing, the cells were transferred to plates containing TH, and allowed to grow for 24 hrs. These plates were then transferred to plates supplemented with erythromycin (EM) (encoded on the vector backbone) at 40 μg/ml and allowed to grow for 24 hrs. The plates were then compared to the previous plates. Ideally, areas of growth on TH plates should show no growth on EM plates and indicate chromosomal integration via a double crossover event. However, these events were not observed. Instead, some areas of growth on TH plates did show growth on EM plates, but growth was delayed 12-24 hours. These regions could be indicative of chromosomal integration via a single integration event. In this event, the entire plasmid is incorporated into the chromosome, thus conferring both TH and EM resistance. However, only one copy of the EM resistance cassette under a weak promoter is present, so that growth under EM is slow. These regions were streaked again on plates supplemented with TH, allowed to grow for 24 hrs, and replica plated onto EM plates to confirm phenotype.

Confirming gene disruption mutants. Single integration gene disruption mutants were confirmed by PCR amplification as described below and PCR amplification of the entire region of integration was completed and sequenced as described in U.S. Patent Pub. No. 2010/0047890. Sequencing primers are given in Table 2.

TABLE 2

| SEQ ID | Sequence | | |
|---|---|---|---|
| NO | Name | Sequence (5′-3′) | Description |
| 1 | SpoIIE-F | GCACTGAGTTCATTTGCTATAAGTAGAGTT | Forward primer to |
| | | | amplify a |
| | | | portion of CAC3205 |
| | | | from C. |
| | | | acetobutylicum |
| | | | ATCC824 |
| | | | genomic DNA |
| 2 | SpoIIE-R | GCTGCTCCTGCTGAACTTC | Reverse primer to |
| | | | amplify a |
| | | | portion of CAC3205 |
| | | | from C. |
| | | | acetobutylicum |
| | | | ATCC824 |
| | | | genomic DNA |
| 3 | SpoIIE-KO-F | CCGCTACCAAGTCAAGAAGCTTTCA | Forward primer to |
| | | | confirm spoIIE |
| | | | gene disruption |
| 4 | SpoIIE-KO-R | ACTCCTGCAGTAATCCACTTTCCAA | Reverse primer to |
| | | | confirm spoIIE |
| | | | gene disruption |
| 5 | CM/TH-F | GGAATGGCGTGTGTGTTAGCCAAA | Forward primer to |
| | | | amplify CM/TH |
| | | | antibiotic cassette |
| 6 | CM/TH-R | TCACACAGGAAACAGCTATGACCA | Reverse primer to |
| | | | amplify CM/TH |
| | | | antibiotic cassette |

Results from SpoIIE disruption mutants. Numerous putative single integration gene disruption mutants resolved following the replica plating protocol. Two large regions displayed the required growing pattern (i.e. growth on TH and delayed growth on EM). Two colonies were selected from each region, so that a total of four colonies were selected for confirmation. Colony PCR was performed to determine the orientation and type of integration using two sets of primers: (1) SpoIIE-KO-F (SEQ ID NO 3) and CM/TH-R (SEQ ID NO 6); and (2) CM/TH-F (SEQ ID NO 5) and SpoIIE-KO-R (SEQ ID NO 4). Refer to FIGS. 1-3 for a schematic explanation and actual PCR results.

In the case of no integration, no PCR product should be generated with either primer set since the TH marker should not be present. If integration occurred through the first region of homology, primer set 1 should produce about 1705 bp product, including the 5′ flanking region of the chromosome, the first region of homology, and the TH marker.

Theoretically, primer set 2 could also produce a product, but it would be >5000 bp, including the TH marker, the second region of homology, the vector backbone, the 3′ coding region up to the point where the first region of homology incorporated, and the 3′ flanking region on the chromosome. If integration occurred through the second region of homology, primer set 2 should produce about 1475 bp product, including the TH marker, the second region of homology, and the 3′ flanking region of the chromosome. As before, theoretically, primer set 1 could also produce a product >5000 bp, including the 5′ flanking region on the chromosome, the coding region up to where the second region of homology ends, the vector backbone, the first region of homology, and the TH marker. The >5000 bp products would not be amplified though since the smaller PCR products would out-compete them for the dNTPs and the extension time is not long enough to produce them. Though unlikely, if a double crossover did occur, both primer sets would give a product.

The PCR reactions were run on the above mentioned four. Two mutants gave a clear band for primer set 2, while another mutant gave an unclear band and another no band. No bands were seen for primer set 1. SpoIIEK020-A was used for further confirmation, which was one of the mutants that rendered PCR product from primer set 2.

In order to definitively confirm SpoIIEK020-A, the integration region is PCR amplified from the flanking chromosome primers and sequenced. Sequencing results prove a single integration through the second region of homology. SpoIIEK020-A will be referred to herein as SpoIIEKO.

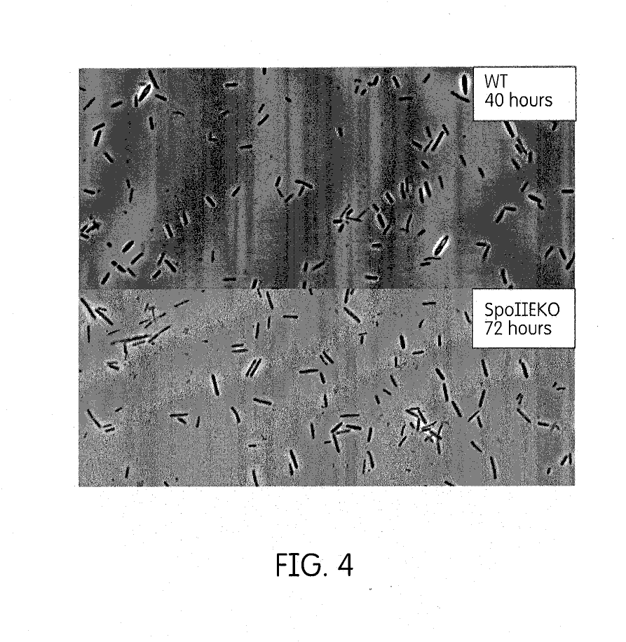

Morphology results from SpoIIEKO. Based on the Bacillus subtilis model, silencing expression of SpoIIE, and hence sigF, should arrest sporulation. To determine if SpoIIEKO differentiates, phase-contrast microscopy was used to observe a culture of SpoIIEKO. In phase-contrast microscopy, free spores are characterized by phase-bright circles, and cells forming a spore have a phase-bright region within the cell. Even at 72 hours, no free spores or spore-forming phenotypes were observed for the SpoIIEKO culture (FIG. 4). The only spore differentiation activity that can be seen is the condensing of DNA at one or both ends of the cell. This is the first step of spore differentiation, usually followed by an asymmetric septation. However, this septation event is never observed for SpoIIEKO, thus confirming that by silencing spoIIE expression, spore differentiation has been arrested.

Additionally, spore viability assays were performed. Cells from a growing culture were serially diluted and plated with and without chloroform treatment at different time points. Only spores would survive the chloroform treatment. After 115 hours, the viable cell count in the wild-type (WT) culture was nearly equal to the spore count, suggesting that all colony forming units were from sporulated cells. However, no SpoIIEKO cells survived the chloroform treatment, strongly suggesting that SpoIIEKO cells do not sporulate. Table 3 below shows the viable cell and viable spore estimates for a WT and SpoIIE KO (20A) at 4 time points and shows that as the WT culture ages, the viable cell count becomes nearly equal to the spore count. For mutant 20A, no cells survived the chloroform treatment thereby leading to the conclusion the SpoIIE deficient cells do not sporulate.

TABLE 3

| | Viable Cell Estimates per ml | | Viable Spore Estimates per ml | |
|---|---|---|---|---|
| Hours | WT | SpoIIEKO | WT | SpoIIEKO |
| 20 | 10⁵ > x > 10⁴ | 2.04 × 10⁸ | 7.05 × 10² | 0 |
| 60 | 1.37 × 10³ | ~10⁵ | 1.05 × 10³ | 0 |
| 90 | 1.33 × 10³ | 3 × 10⁵ | 1.2 × 10³ | 0 |
| 115 | 9.1 × 10² | 9.2 × 10⁴ | 8.65 × 10² | 0 |

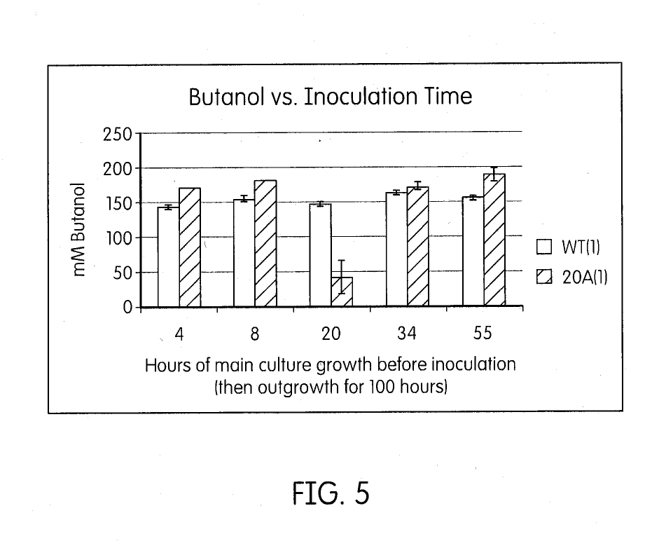

SpoIIEKO's enhanced butanol production dependent upon age of inoculum. SpoIIEKO cultures inoculated with a typical inoculum, i.e., exponentially growing cells, produced at most about 45 mM of butanol, which is substantially less than typical WT culture productivity of 150-180 mM. However, it was surprisingly found that if a genetically older culture is used as the inoculum (i.e., a culture in the post-exponential phase of growth), SpoIIEKO can achieve enhanced butanol production compared to WT cultures. Static flasks for SpoIIEKO (2 biological replicates) and WT (2 biological replicates) were inoculated with typical inoculums of exponentially growing cells. These flasks were then used to inoculate 35 ml cultures in 50 ml tubes (with 3 technical replicates for each biological replicate) at 4 hrs, 8 hrs, 20 hrs, 34 hrs, and 56 hrs. Tubes were grown for 120 hrs, and end-point product concentrations were determined. WT cultures displayed no dependence on inoculation time, while the SpoIIEKO cultures did (see FIG. 5). When inoculated from a 20 hr old culture (a typical exponential phase culture), the SpoIIEKO culture only produced 42 mM of butanol. However, at all other inoculation times (i.e., post-20 hours of growth and post-exponential phase of growth), SpoIIEKO produced close to WT levels of butanol, and from the inoculum at 56 hrs (i.e., an inoculum in the stationary phase of growth), actually produced 17% more butanol than WT (see FIG. 5). Table 4 below shows glucose consumed after 120 hours of growth for SpoIIEKO and WT, and indicates that butanol production for WT is independent of inoculation time.

TABLE 4

Glucose consumed (mM)

| | SpoIIEKO | |
|---|---|---|
| Inoculation time | (n = 2) | WT (n = 2) |
| 4 hr | 340 (±1) | 282 (±3) |
| 8 hr | 328 (±1) | 292 (±6) |
| 20 hr | 138 (±24) | 295 (±4) |
| | 326 (±7) | 311 (±5) |
| 34 hr | 326 (±7) | 311 (±5) |
| | 326 (±7) | 311 (±5) |
| 56 hr | 351 (±10) | 310 (±3) |

Although the present invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the appended claims.

What is claimed is:

1. A Clostridium cell, wherein expression of the SpoIIE gene of said Clostridium cell is silenced.

2. The Clostridium cell of claim 1, wherein an asymmetric septation event in said Clostridium cell does not occur throughout the life of the cell in culture.

3. The Clostridium cell of claim 2, wherein said Clostridium cell is selected from the group consisting of C. acetobutylicum, C. beijerinckii, C. saccharoperbutylacetonicum, and C. madisonii.

4. The Clostridium cell of claim 1, wherein sporulation by said Clostridium cell is abolished.

5. The Clostridium cell of claim 1, wherein said Clostridium cell exhibits increased solvent production relative to a wild-type Clostridium cell of the same strain.

6. The Clostridium cell of claim 5, wherein said solvent is selected from the group consisting of butanol, butyric acid, acetoin, butanediol, and propanol.

7. The Clostridium cell of claim 5, wherein said solvent comprises butanol.

8. The Clostridium cell of claim 5, wherein said Clostridium cell exhibits at least about 15% increased solvent production relative to a wild-type Clostridium cell of the same strain.

9. The Clostridium cell of claim 1, wherein said Clostridium cell is used for industrial-scale production of a chemical product selected from the group consisting of butanol, butyric acid, acetoin, butanediol, and propanol.

10. A method for abolishing sporulation in a Clostridium cell comprising silencing the expression of the SpoIIE gene of the cell, wherein sporulation by the cell is abolished.

11. The method of claim 10, comprising silencing the expression of the SpoIIE gene by homologous recombination.

12. The method of claim 11, comprising the step of transforming the cell with a vector comprising a nucleic acid that disrupts the function of the SpoIIE gene following homologous recombination, wherein the nucleic acid integrates into the genome of the Clostridium cell.

13. The method of claim 12, wherein the nucleic acid comprises a mutated SpoIIE gene.

14. The method of claim 13, wherein the plasmid comprises a resolvase gene and the resolvase gene is expressed in the Clostridium cell.

15. A method for increasing solvent production in bacterial cells, comprising inoculating a medium with an inoculum of the bacterial cells, wherein expression of a sporulation gene in the bacterial cells is inhibited or silenced, and wherein the bacterial cells of the inoculum are in a post-exponential phase of growth prior to inoculation.

16. The method of claim 15, wherein the bacterial cells of the inoculum are in a stationary phase of growth prior to inoculation.

17. The method of claim 15, wherein the inoculum has been growing for at least 20 hours prior to inoculation.

18. The method of claim 15, wherein the bacterial cells of the inoculum have not committed to endospore formation prior to inoculation.

19. The method of claim 15, wherein the bacterial cells are Clostridium cells and expression of the SpoIIE gene in the Clostridium cells is silenced.

20. A method for producing a chemical product, said method comprising contacting a Clostridium cell in accordance with claim 1 with a feedstock.
